(19) United States
(12) Patent Application Publication
(10) Pub. No.: US 2015/0087657 A1
(43) Pub. Date: Mar. 26, 2015

(54) MULTICOMPONENT CRYSTALLINE SYSTEM OF VORICONAZOLE WITH FUMARIC ACID

(71) Applicant: BASF SE, Ludwigshafen (DE)

(72) Inventors: Andreas Hafner, Gelterkinden (CH); Tobias Hintermann, Therwil (CH); Fritz Blatter, Reinach (CH); Eva Roedel, Basel (CH)

(73) Assignee: BASF SE, Ludwigshafen (DE)

(21) Appl. No.: 14/360,799

(22) PCT Filed: Dec. 3, 2012
(86) PCT No.: PCT/IB12/56907

§ 371 Date: May 27, 2014

Related U.S. Application Data
(60) Provisional application No. 61/567,136, filed on Dec. 6, 2011.

(30) Foreign Application Priority Data
Dec. 6, 2011 (EP) 11192058.3

(51) Int. Cl. A61K 31/506; A61K 31/194

(52) U.S. Cl. CPC A61K 31/506; A61K 31/194
USPC 514/256

(57) ABSTRACT

A novel solid form of Voriconazole comprises the active ingredient (2R,3S)-2-(2,4-difluorophenyl)-3-(5-fluoropyrimidin-4-yl)-1-(1H-1,2,4-triazol-1-yl)butan-2-ol and fumaric acid. The solid composition comprising the two components shows improved properties such as water solubility, crystallization behavior and stability.

The present invention relates to a multicomponent system comprising voriconazole and fumaric acid, to pharmaceutical preparations comprising said system, and specifically to a homogenous crystalline phase (cocrystal) comprising voriconazole and fumaric acid. The invention also relates to processes for preparing said multicomponent system and crystalline phase. The invention also relates to compositions comprising said multicomponent system or crystalline phase and a pharmaceutically acceptable carrier, and to methods of using said multicomponent system or crystalline phase to treat a disease condition wherein control of aggressive fungi (e.g. aspergilli, candida, scedosporium, fusarium) is beneficial.

Voriconazole is the compound (2R,3S)-2-(2,4-difluorophenyl)-3-(5-fluoropyrimidin-4-yl)-1-(1H-1,2,4-triazol-1-yl)butan-2-ol characterized by the following chemical formula:

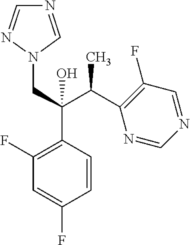 [SMILES: C[C@@H](c1ncncc1F)[C@](O)(Cn1cncn1)c1ccc(F)cc1F]

Preparation of Voriconazole has been described inter alia in WO 06/065726, leading to polymorphic forms A and B as well as the amorphous form.

Since the application of Voriconazole often requires the preparation of an aqueous solution, while the compound shows very low solubility in water, an improvement of its crystalline forms is desirable. WO 09/053993 describes a crystalline oxalate of Voriconazole. WO 11/020605 describes a Voriconazole-cyclodextrin complex.

It has now been found that fumaric acid (i.e. trans-2-butenedioic acid) forms co-crystals with Voriconazole showing an improved morphology, while other excipients like maleic acid or lactic acid fail to do so. Specifically, the present composition, which comprises Voriconazole together with fumaric acid within the same solid phase, shows an improved solubility in aqueous environments.

### SUMMARY OF THE INVENTION

The invention provides a novel solid form of Voriconazole characterized by a content of fumaric acid and, consequently, novel pharmaceutical formulations containing this form. The invention further provides a novel crystalline form characterized by containing Voriconazole and fumaric acid within the same crystalline phase, and processes for manufacture thereof.

Crystalline forms often show desired physical and/or biological characteristics, which differ from other solid forms and may assist in the manufacture or formulation of the active compound and/or contribute to the purity levels and uniformity required for regulatory approval. The present solid form, especially crystalline form, may possess improved pharmacological characteristics, for example, improved bioavailability, thus offering enhanced possibilities to modulate and design improved drug products.

### DETAILED DESCRIPTION OF THE INVENTION

The solid composition of the invention generally is a composite comprising two components, which are Voriconazole and fumaric acid within one single phase.

The present solid form generally contains about 0.5 to 1.5 molar parts of fumaric acid on 1 molar part of (2R,3S)-2-(2,4-difluorophenyl)-3-(5-fluoropyrimidin-4-yl)-1-(1H1,2,4-triazol-1-yl)butan-2-ol [Voriconazole].

More specifically the solid phase contains the 2 components in a ratio of about 1:1 (i.e. 1:1 adduct), with common variations as known from crystalline phases, e.g. due to vacancy defects, interstitial defects, and/or minor impurities (e.g. by other acid or base components). Preferred molecular ratios thus range from 0.9 to 1.1 molar parts, especially from 0.95 to 1.05 molar parts, of fumaric acid on 1 molar part of Voriconazole.

The solid composition of the invention may be used during preparation of the medicament comprising Voriconazole, e.g. for the purpose of purification, as well as in the final application form. The single phase form comprising Voriconazole and fumaric acid as a molecular crystal (co-crystal) may be added as such, or may be formed in situ.

The invention thus includes

i) a multicomponent molecular crystal containing Voriconazole and fumaric acid;
ii) a multicomponent molecular crystal containing 0.5 to 1.5 molar parts, preferably 0.9 to 1.1 molar parts, and much preferred a 1:1 adduct, of Voriconazole and fumaric acid;
iii) a solid form as defined under i-ii consisting essentially of Voriconazole and fumaric acid;
iv) a molecular crystal, especially co-crystal, of Voriconazole and fumaric acid.

Preferred solid form may be further characterized by its high crystallinity. While showing a improved solubility, the present solid form further provides good stability, and advantages in processing due to its good crystallization properties (crystallisation from alcohol or water without co solvents).

Voriconazole and fumaric acid are present in the same solid phase, preferably in the same crystalline phase, i.e. forming a co-crystal. The invention thus further pertains to a novel crystalline form of Voriconazole, which crystalline form is characterized by containing fumaric acid within its crystalline structure, e.g. in amounts as indicated above. A preferred novel crystalline form generally exhibits a characteristic X-ray powder diffraction pattern with characteristic peaks expressed in d-values (Å):

11.6 (s), 7.8 (w), 6.0 (w), 5.72 (w), 5.45 (vs), 5.20 (s), 5.03 (w), 4.83 (s), 4.66 (s), 4.47 (m), 4.19 (w), 4.08 (w), 3.87 (m), 3.81 (s), 3.69 (w), 3.59 (s), 3.55 (m), 3.47 (s), 3.41 (w), 3.36 (w), 3.23 (w), 3.22 (w), 3.12 (w), 3.05 (m), 3.01 (m), 2.91 (w), 2.86 (w), 2.74 (w), 2.62 (w), 2.59 (w), 2.54 (w).

More specifically, the present invention comprises a crystalline form of Voriconazole and fumaric acid, which exhibits a characteristic X-ray powder diffraction pattern with characteristic peaks as shown in table 2 further below, especially as shown in FIG. 1.

Here and in the following, the abbreviations in brackets mean: (vs)=very strong intensity; (s)=strong intensity; (m)=medium intensity; (w)=weak intensity. (vw)=veryweak, intensity.

Another object of the invention is a process for the preparation of a solid composition comprising the compound (2R,3S)-2-(2,4-difluorophenyl)-3-(5-fluoropyrimidin-4-yl)-1-(1H-1,2,4-triazol-1-yl)butan-2-ol (Voriconazole) and fumaric acid, especially as described above, which process comprises the step of contacting Voriconazole with fumaric acid.

The process may be carried out by mixing the components Voriconazole and fumaric acid, e.g. at a temperature ranging from 0 to 180° C., especially 0-100° C., using suitable means such as milling of the solids or stirring of liquids. Without presence of a solvent, the process may be carried out, for example, by dry milling, compacting, and or heating, for example to temperatures close to or above the melting point of Voriconazole, e.g. under nitrogen. Typical temperatures for the contacting or mixing step are from the range 0-180° C. Without presence of solvents, temperatures typically are higher, e.g. 80-180° C., or 90-150° C.

Preferably, the contacting or mixing step is carried out in the presence of a solvent at temperatures within the liquid range of the chosen solvent under normal pressure, e.g. 0-100° C., or 10-80° C.

The solvent used may be water or, preferably, a water miscible organic solvent such as an alcohol (e.g. methanol, ethanol, propanol, butanol), or an ester (such as ethyl acetate, methyl acetate), ethers such as methyl-tert.butylether, or an aliphatic ketone (e.g. acetone, methyl ethyl ketone), or mixture of such solvents, or such a solvent with water. Of special preference are solvents selected from alcohols and water, especially ethanol. Solutions or suspensions used for the contacting/mixing step preferably cornprise solutions.

Thus, the components Voriconazole and fumaric acid may be mixed conveniently in presence of a solvent to obtain a solution or suspension, and the solvent is subsequently removed, e.g. by filtration and/or evaporation and/or drying.

The contacting/mixing step may advantageously be carried out using the educts Voriconazole and/or fumaric acid as finely ground powders, or pre-suspended in the solvent, or in dissolved state. One way of forming the solid composition of the invention thus would be a recrystallization wherein each of the solid educts is dissolved or suspended in the solvent, both educts are combined and mixed, and the mixture then is cooled e.g. to 0-25° C. in order to initiate or improve precipitation before removal of the solvent as noted above.

Advantageously, seed crystals of the present cocrystalline form comprising both components are added before final removal of the solvent.

The concentration of Voriconazole may range from 0.1 to about 300 mg/ml of solvent (especially including alcohol such as ethanol), preferably from 5 to 200 mg/ml.

The process is preferably carried out in the temperature range 15-50° C., for example at ambient temperature. In a preferred process, contacting/mixing is carried out at a temperature from the range 20-60° C. or the mixture is heated to a temperature from said range, e.g. about 50° C. The suspension thus tempered is then preferably cooled before removal of solvent. In a preferred process, the step is accompanied by seeding with the present co-crystal (e.g. 1-10% b.w. of the total amount of Voriconazole) at a temperature of about 20-50° C.

Ambient temperature means in the context of the invention a temperature range at room temperature, comprising 20 to 30° C. and preferably about 22 to 25° C. When using solvents, the present solid composition usually is isolated by filtering off the crystals and drying, e.g. in vacuum, an inert gas flow or both at ambient temperature, or elevated temperatures up to 60° C.

The present solid composition, especially the co-crystal, is thermodynamically stable and can be dried at elevated temperatures, e.g. at 25-80° C., and is obtained as a fine powder with typical particle size distributions with the median size between 1 and 50 preferably between 1 to 10 μm. This particle size range ensures a fast dissolution profile, while retaining the favorable handling properties in the formulation process.

The solid form of the invention may be used in pharmaceutical compositions in the same way as other forms of Voriconazole previously known. Additionally, present solid composition may be employed as an intermediate or starting material to produce the pure active ingredient, e.g. in form of crystal form A or B.

The present invention is also directed to a pharmaceutical composition comprising a solid form of Voriconazole and containing fumaric acid, or especially the present co-crystal comprising Voriconazole and fumaric acid, or hydrates thereof, and a pharmaceutically acceptable carrier or diluent.

The amount of solid (especially crystalline) forms Voriconazole and fumaric acid and hydrates thereof substantially depends on type of formulation and desired dosages during administration time periods. The amount in an oral formulation may be, for example, from 1 to 500 mg, typically from about 40 to 200 mg.

Formulations may be solid formulations such as capsules, tablets, pills and troches, or liquid formulations such as aqueous suspensions, elixirs, syrups, infusions, intravenous drips. Solid and liquid formulations encompass also incorporation of the present solid form, especially co-crystal, into liquid or solid food.

The solid forms according to the invention may be directly used as powders (micronized particles), granules, suspensions or solutions, or they may be combined together with other pharmaceutically acceptable ingredients in admixing the components and optionally finely divide them, and then filling capsules, composed for example from hard or soft gelatine, compressing tablets, pills or troches, or suspend or dissolve them in carriers for suspensions, elixirs and syrups. Coatings may be applied after compression to form pills.

Pharmaceutically acceptable ingredients are well known for the various types of formulation and may be for example binders such as natural or synthetic polymers, excipients, lubricants, surfactants, sweetening and other flavouring agents, coating materials, preservatives, dyes, thickeners, adjuvants, antimicrobial agents and carriers for the various formulation types.

Examples for binders are gum tragacanth, acacia, starch, gelatine, and biological degradable polymers such as homo- or co-polyesters of dicarboxylic acids, alkylene glycols, polyalkylene glycols and/or aliphatic hydroxyl carboxylic acids; homo- or copolyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, und natural polymers like chitosan, carragenan or hyaluronic aid.

Examples for excipients are phosphates such as dicalcium phosphate.

Examples for lubricants are natural or synthetic oils, fats, waxes, or fatty acid salts like magnesium stearate.

Surfactants may be anionic, anionic, amphoteric or neutral. Examples for surfactants are lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Examples for sweetening agents are sucrose, fructose, lactose or aspartam.

Examples for flavouring agents are peppermint, oil of wintergreen or fruit flavours like cherry or orange flavour.

Examples for coating materials gelatine, wax, shellac, sugar or biological degradable polymers.

Examples for preservatives are methyl or propylparabens, sorbic acid, chlorobutanol, phenol and thimerosal.

Examples for adjuvants are fragrances.

Examples for thickeners are synthetic polymers, fatty acids and fatty acid salts and esters and fatty alcohols.

Examples for liquid carriers are water, alcohols such as ethanol, glycerol, propylene glycol, liquid polyethylene glycols, triacetin and oils. Examples for solid carriers are talc, clay, microcrystalline cellulose, silica, alumina and the like.

The formulation according to the invention may also contain isotonic agents, such as sugars, buffers or sodium chloride.

The solid forms according to the invention may also be formulated as effervescent tablet or powder, which disintegrate in an aqueous environment to provide a drinking solution.

A syrup or elixir may contain the cocrystal of the invention, sucrose or fructose as sweetening agent a preservative like methylparaben, a dye and a flavouring agent.

The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the art of pharmacy.

Dosage forms include solid dosage forms, like tablets, powders, capsules, suppositories, sachets, troches and losenges as well as liquid suspensions and elixirs. While the description is not intended to be limiting, the invention is also not intended to pertain to true solutions of Voriconazole whereupon the properties that distinguish the solid forms of Voriconazole are lost. However, the use of the novel forms to prepare such solutions is considered to be within the contemplation of the invention.

Capsule dosages, of course, will contain the solid composition within a capsule which may be made of gelatin or other conventional encapsulating material. Tablets and powders may be coated. Tablets and powders may be coated with an enteric coating. The enteric coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Slow release formulations may also be prepared from the crystal form according to the invention in order to achieve a controlled release of the active agent in contact with the body fluids in the gastro intestinal tract, and to provide a substantial constant and effective level of the active agent in the blood plasma. The crystal forms may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

The solid composition of the invention is also useful for administering a combination of therapeutic effective agents to an animal. Such a combination therapy can be carried out in using at least one further therapeutic agent which can be additionally dispersed or dissolved in a formulation.

The solid composition of this invention and its formulations respectively can be also administered in combination with other therapeutic agents that are effective to treat a given condition to provide a combination therapy.

The solid composition and the pharmaceutical composition according to the invention are highly suitable for effective treatment of disorders in connection with fungal infections or wherein control of fungi is beneficial.

An object of the invention is also a therapeutic method for producing an antifungal effect in a mammal comprising administering to a mammal in need of such therapy, an effective amount of the present solid composition or composite containing Voriconazole and fumaric acid, and/or hydrates thereof.

The multicomponent crystal of the invention may be used as single component or as mixtures with other solid forms, which may be crystalline or amorphous.

As to the novel multicomponent crystal of Voriconazole it is preferred that these contain 25-100% by weight, especially 50-100% by weight, based on the total amount of Voriconazole. Preferably, such an amount of the novel multicomponent crystal forms of Voriconazole is 75-100% by weight, especially 90-100% by weight. Highly preferred is an amount of 95-100% by weight.

Another object of the invention is a method of delivering a solid form of (2R,3S)-2-(2,4-difluorophenyl)-3-(5-fluoropyrimidin-4-yl)-1-(1H-1,2,4-triazol-1-yl)butan-2-ol and/or hydrates thereof to a host, which method comprises administering to a host an effective amount of the solid composition of the invention, especially the present co-crystal.

A further object of the invention is the use of a solid composition of the invention as described above and in the below examples for the manufacture of a medicament useful in the treatment of disorders wherein control of fungi is beneficial, and especially useful in the treatment and control of aspergilli, candida, scedosporium, fusarium.

The object of the invention includes the solid composition according to the invention for use in medical therapy.

The following examples illustrate the invention.

Wherever noted, room temperature (r.t.) depicts a temperature from the range 22-25° C.; over night means a period of 12 to 15 hours; percentages are given by weight, if not indicated otherwise. The unit Angstroem (Å) denotes the distance 10⁻¹⁰ m.

### ABBREVIATIONS

DMSO dimethyl sulfoxide
HPLC high pressure liquid chromatography
NMR nuclear magnetic resonance
FTIR Fourier-transformation infrared spectrometry
Mw molecular weight
m.p. melting point
r.h. relative humidity (air, if not indicated otherwise)
TG thermogravimetry
DSC differential scanning calorimetry
v/v volume by volume
PXRD Powder X-ray diffraction

### vrx Voriconazole

### Instrumental

Powder X-ray diffraction (PXRD): Measurements are carried out with a Stoe Stadi P diffractometer equipped with a Mythen1K Detector; Cu—K-alpha radiation. Measurement conditions: transmission; 40 kV and 40 mA tube power; curved Ge monochromator; 0.02° step size, 12 s step time, 1.5-50.5° 2θ scanning range; detector mode: step scan; 1° detector step. Sample preparation: 10 to 20 mg sample is placed between two acetate foils and mounted into a Stoe transmission sample holder. The sample is rotated during the measurement.

### Thermogravimetry Coupled to Infrared Spectroscopy (TG-FTIR):

The thermogravimetric measurements are carried out with a Netzsch ThermoMicrobalance TG 209 coupled to a Bruker FTIR Spectrometer type Vector 22 (aluminum crucibles with micro pinhole, N2 atmosphere, heating rate 10 K/min, range 25° C. to 250° C.).

### Differential Scanning Calorimetry (DSC):

DSC is carried out using a Perkin Elmer DSC-7. Samples are placed into gold crucibles sealed under nitrogen. The measurements are performed with a heating rate of 10 or 20° C. min⁻¹ over the temperature range from −50° C. to about 200° C.

### FT-Raman Spectroscopy:

Raman spectra are recorded with a Bruker RFS100 Raman spectrometer equipped with a germanium detector and a Nd:YAG laser with an excitation wavelength of 1064 nm, each recording using a few milligrams of material pressed into aluminum sample holders. Spectra in the range of 50-3500 cm⁻¹ and with a resolution of 2 cm⁻¹ are detected with a laser power of 300 mW. 64 scans are accumulated. 1H-NMR:

The 1H-NMR spectra are recorded on a Bruker DPX 300 spectrometer.

Solvent: DMSO-d6.

Experimental

Solvents: For all experiments, Fluka or Sigma Aldrich grade solvents are used. Selected solvents are dried using 3 or 4 Å molecular sieves.

Characterization of Voriconazole (Starting Material):

A commercial sample of Voriconazole is characterized by powder X-ray diffraction (see Table 1), H-NMR and C13-NMR spectroscopy, Raman spectroscopy, TG-FTIR and DSC.

TABLE 1
Educt Sample Identification

| | |
|---|---|
| Name | Voriconazole |
| Formula | C₁₆H₁₄F₃N₅O₂ |
| Mw (g/mol) | 349.3 |
| DSC | m.p. 133° C., □H = 97 J/g |
| PXRD | as of Form B (FIG. 4 of WO 06/65726) |

Crystallization experiments: The crystallization experiments are performed in Supelco glass vials using magnetic stirrers.

### Example 1

### Preparation of Cocrystal with Fumaric Acid

108.7 mg of vrx and 34.1 mg of fumaric acid are dissolved in 5 ml of ethanol; then the solvent is evaporated under a slight flow of nitrogen at room temperature. To the dry residue, 0.4 ml ethanol is added and the mixture is stirred at room temperature for two days or until a suspension with crystalline material is obtained. The solid material is separated by filtration and dried under vacuum (10 mbar) at room temperature for 4 hours. The crystalline material obtained is investigated by powder X-ray diffraction and 1H-NMR. NMR spectroscopy suggests that a material with a molar ratio of vrx to fumaric acid of 1:1 is obtained. The PXRD pattern (as shown in FIG. 1) is characteristic for a co-crystal of vrx and fumaric acid. The peak locations of the PXRD pattern are provided in table 2.

### Example 2

### Preparation of Cocrystal with Fumaric Acid

401 mg of vrx and 133 mg of fumaric acid are dissolved in 7 mL of ethanol by heating to reflux temperature. The mixture is allowed to cool to room temperature and stirred at room temperature for about one hour. About 50% of the solvent is evaporated under a slight flow of nitrogen (about 30 ml per min), about 10 mg of vrx-fumaric acid co-crystal seeds of example 1 are added. 4 ml isopropanol is added and the resulting suspension is stirred at room temperature for two days before the obtained solid is separated by filtration and dried under vacuum (about 10 mbar) at room temperature for about one hour. The obtained crystalline material is investigated by powder X-ray diffraction, FT-Raman spectroscopy, TG-FTIR, light microscopy and H-NMR. NMR spectroscopy suggests that a material with a molar ratio of vrx to fumaric acid with a 1:1 ratio is obtained. The PXRD pattern (identical with FIG. 1) and the Raman spectrum are characteristic for a co-crystal of vrx and fumaric acid. The peak locations of the PXRD pattern are as shown in table 2; peak locations of the Raman spectrum are provided in table 3. Light microscopy reveals that a uniform sample with small particle sizes is produced.

TABLE 2
PXRD peak locations for vrx - fumaric acid co-crystal.

| | | qualitative |
|---|---|---|
| d-spacinq | | relative |
| [Å] | angle 2θ | intensity |
| 11.6 | 7.6 | s |
| 10.1 | 8.8 | vw |
| 8.7 | 10.2 | vw |
| 7.8 | 11.4 | w |
| 7.0 | 12.7 | vw |
| 6.0 | 14.7 | w |
| 5.78 | 15.3 | vw |
| 5.72 | 15.5 | w |
| 5.45 | 16.3 | vs |
| 5.20 | 17.0 | s |
| 5.03 | 17.6 | w |
| 4.83 | 18.4 | s |
| 4.66 | 19.0 | s |
| 4.47 | 19.8 | m |
| 4.33 | 20.5 | vw |
| 4.19 | 21.2 | w |
| 4.10 | 21.6 | vw |
| 4.08 | 21.8 | w |
| 3.89 | 22.9 | vw |
| 3.87 | 23.0 | m |
| 3.81 | 23.3 | s |
| 3.69 | 24.1 | w |
| 3.61 | 24.6 | vw |
| 3.59 | 24.8 | s |
| 3.55 | 25.1 | m |
| 3.52 | 25.3 | vw |
| 3.47 | 25.6 | s |
| 3.41 | 26.1 | w |
| 3.36 | 26.5 | w |
| 3.23 | 27.6 | w |
| 3.22 | 27.7 | w |
| 3.12 | 28.6 | w |
| 3.05 | 29.2 | m |
| 3.01 | 29.6 | m |
| 2.99 | 29.8 | vw |
| 2.91 | 30.7 | w |
| 2.89 | 30.9 | vw |
| 2.86 | 31.2 | w |
| 2.82 | 31.7 | vw |
| 2.80 | 32.0 | vw |
| 2.76 | 32.4 | vw |
| 2.74 | 32.6 | w |
| 2.65 | 33.8 | vw |
| 2.63 | 34.1 | vw |
| 2.62 | 34.2 | w |
| 2.59 | 34.6 | vw |
| 2.59 | 34.7 | w |
| 2.57 | 34.9 | vw |
| 2.54 | 35.4 | w |

TABLE 3
Raman peak table for the vrx - fumaric acid co-crystal
(intensity in arbitary units).

| wavenumber | intensity |
|---|---|
| 3898 | 7 |
| 3054 | 18 |
| 2994 | 6 |
| 2969 | 10 |
| 2949 | 5 |
| 1702 | 48 |
| 1645 | 7 |
| 1618 | 4 |
| 1600 | 9 |
| 1464 | 6 |
| 1427 | 4 |
| 1377 | 14 |
| 1350 | 4 |
| 1268 | 19 |
| 1256 | 7 |
| 1237 | 5 |
| 1199 | 5 |
| 1159 | 6 |
| 1138 | 12 |
| 1101 | 14 |
| 1064 | 17 |
| 1016 | 13 |
| 971 | 7 |
| 958 | 6 |
| 910 | 8 |
| 879 | 11 |
| 784 | 7 |
| 747 | 9 |
| 730 | 32 |
| 704 | 4 |
| 612 | 6 |
| 590 | 4 |
| 566 | 9 |
| 536 | 14 |
| 513 | 5 |
| 459 | 5 |
| 397 | 8 |
| 377 | 4 |
| 330 | 6 |
| 298 | 10 |
| 239 | 14 |
| 193 | 20 |
| 164 | 6 |
| 106 | 16 |

### Example 3

### Solubility Determination of Vrx and Vrx-Co-Crystals

Solubility determinations of the vrx (free drug substance), the vrx-fumaric acid co-crystal and the vrx-L-tartaric acid co-crystal described in the below comparative example are carried out as follows. To 20 mg of a solid sample, 5.0 ml water is added and the mixture is placed on a laboratory shaker at 500 rpm at 37° C. After 24 hours, a small sample of about 1.0 ml is recovered with a syringe and filtered through a 0.1 micrometer PVDF Millipore filtration unit. This sample is appropriately diluted and the concentration is determined by HPLC.

The results of these solubility tests are shown in table 4 below from which it is readily noted that the aqueous solubility of the fumaric acid co-crystal is about 30% higher than the free drug substance and the L-tartaric acid co-crystal.

TABLE 4
Solubility of Voriconazole and Voriconazole cocrystals

| | Equilibration | Solubility* |
|---|---|---|
| Sample | Time | in mg/mL |
| Voriconazole free drug (comparison) | 24 h | 1.01 |
| Co-crystal with fumaric acid (invention) | 24 h | 1.30 |
| Co-crystal with L-tartaric acid (comp.) | 24 h | 0.93 |

*of Voriconazole

### Comparative Example 1

### Preparation of Cocrystal with Tartaric Acid

a) Preparation of seeding crystals: To 150 mg of vrx 65 mg of L-tartaric acid, 40 microliter of ethanol is added and the mixture is placed into an agate container for a Retsch MM200 ball mill. The mixture is milled at 30 Hz 3×15 min with 15 min breaks between each milling cycle. The product obtained after milling is investigated by FT-Raman spectroscopy and powder X-ray diffraction. Both methods show that a new solid material neither containing free vrx or free L-tartaric acid is obtained. The molar ratio in this new compound is about 1:1.

b) 183.5 mg vrx and 80.1 mg of L-tartaric acid are suspended in 1.0 ml ethanol; the mixture is sonicated for about one minute then heated slightly to dissolve the remaining solid. Thereafter, about 50% of the solvent is evaporated under slight flow of nitrogen (about 30 ml/min) or until a suspension is obtained. The mixture is seeded with about 5 mg of the vrx-L-tartaric acid co-crystal form B according to (a) and sonicated for about one minute. The thick suspension is now diluted with about one ml of heptane, and stirring is continued while the temperature is cycled as follows: 25° C. for one hour, from 25° C. to 40° C. in one hour, kept at 40° C. one hour and cooled to 25° C. within one hour, then again kept at 25° C. for one hour. After one week the solid is separated by filtration, dried under vacuum at room temperature for one hour (10 mbar) and investigated by powder X-ray diffraction, FT-Raman spectroscopy, TG-FTIR, H-NMR and light microscopy.

### Comparative Example 2

### Maleic Acid

To 150 mg of vrx, 50 mg of maleic acid and 40 microliter of ethanol is added. The mixture is placed into an agate container for a Retsch MM200 ball mill. The mixture is milled at 30 Hz 3×15 min with 15 min breaks between each milling cycle. The product obtained after milling is investigated by powder X-ray diffraction. The PXRD pattern is an overlay of both educts (Voriconazole and maleic acid), showing that no cocrystal has been formed.

### BRIEF DESCRIPTION OF FIGURES

FIG. 1: Powder X-Ray Diffraction pattern of the Voriconazole-fumaric acid co-crystal

1. A solid composition comprising (2R,3S)-2-(2,4-difluorophenyl)-3-(5-fluoropyrimidin-4-yl)-1-(1H-1,2,4-triazol-1-yl)butan-2-ol (Voriconazole) and fumaric acid in one common single phase.

2. The solid composition of claim 1 comprising more than 50% by weight of Voriconazole and fumaric acid.

3. The solid composition of claim 1 consisting essentially of Voriconazole and fumaric acid.

4. The solid composition of claim 1, comprising 0.5 to 1.5 molar parts of fumaric acid to 1 molar part of Voriconazole.

5. The solid composition of claim 1, comprising 0.9 to 1.1 molar parts of fumaric acid to 1 molar part of Voriconazole.

6. The solid composition of claim 1, consisting essentially of Voriconazole, fumaric acid, and water, where water is a minor component by weight.

7. The solid composition of claim 1, in a crystalline form of Voriconazole with fumaric acid, and/or hydrates thereof, exhibiting an X-ray powder diffraction pattern with characteristic peaks expressed in d-values (Å): 11.6 (s), 7.8 (w), 6.0 (w), 5.72 (w), 5.45 (vs), 5.20 (s), 5.03 (w), 4.83 (s), 4.66 (s), 4.47 (m), 4.19 (w), 4.08 (w), 3.87 (m), 3.81 (s), 3.69 (w), 3.59 (s), 3.55 (m), 3.47 (s), 3.41 (w), 3.36 (w), 3.23 (w), 3.22 (w), 3.12 (w), 3.05 (m), 3.01 (m), 2.91 (w), 2.86 (w), 2.74 (w), 2.62 (w), 2.59 (w), 2.54 (w).

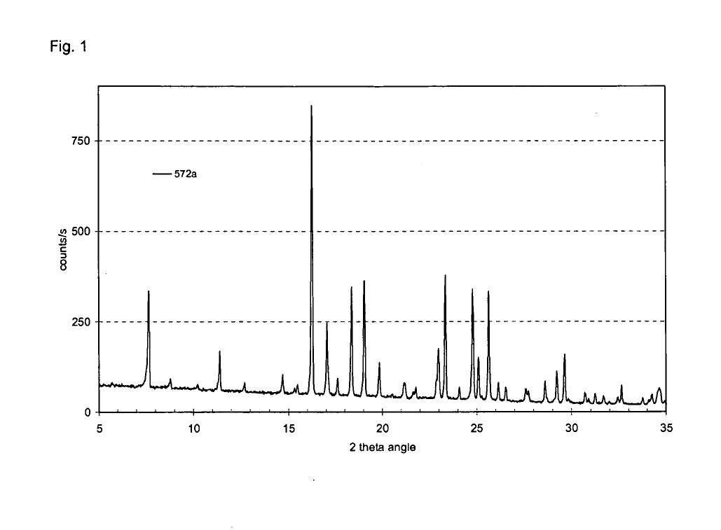

8. The solid composition of claim 1, comprising a crystalline form of Voriconazole with fumaric acid, and/or hydrates thereof, which exhibits a characteristic X-ray powder diffraction pattern substantially as exhibited in FIG. 1.

9. A process for preparing the solid composition of claim 1, the process comprising contacting Voriconazole with fumaric acid.

10. The process of claim 9, wherein the contacting comprises mixing the Voriconazole and fumaric acid at a temperature ranging from 0 to 180° C., with or without the presence of a solvent.

11. The process of claim 9, wherein the contacting comprises mixing the Voriconazole and fumaric acid in the presence of a solvent to obtain a solution or suspension, and the process further comprises subsequently removing the solvent from the solution or suspension.

12. A pharmaceutical composition comprising the solid composition of claim 1 and a pharmaceutically acceptable carrier or diluent.

13. A method for producing a solid application form of a medicament comprising Voriconazole, the method comprising adding Voriconazole and fumaric acid to the medicament.

14. (canceled)
